(19) United States
(12) Patent Application Publication
(10) Pub. No.: US 2011/0130821 A1
(43) Pub. Date: Jun. 2, 2011

(54) DEVICE FOR TREATING A BLOOD FLOW CONDUIT

(76) Inventor: Witold Styrc, Luxembourg (LU)

(21) Appl. No.: 12/996,160

(22) PCT Filed: Jun. 4, 2009
(86) PCT No.: PCT/FR09/51055

§ 371 Date: Jan. 26, 2011

(30) Foreign Application Priority Data
Jun. 5, 2008 (FR) 0853732

(51) Int. Cl. A61F 2/84

(52) U.S. Cl. 623/1.11

(57) ABSTRACT

The present invention relates to a device for processing a blood circulation conduit comprising: at least one endoprosthesis deployable between a retracted state and a dilated state; a stent for mounting the endoprosthesis, the stent including a body extending longitudinally between a proximal end region and a distal end region, the stent being fitted for the insertion, positioning, and deployment of the endoprosthesis in the blood circulation conduit, the stent including a releasable means for retaining the endoprosthesis on the stent. The distal end region of the body of the stent is capable of changing the shape thereof between a substantially straight configuration and an inwardly curved configuration, and the device comprises a controlling means for changing the shape of said distal end region between the straight configuration thereof and the inwardly curved configuration thereof.

The present invention relates to a device for treating a blood flow conduit, of the type comprising:

at least one endoprosthesis deployable between a retracted state and a dilated state;

a support catheter for supporting the endoprosthesis, the support catheter comprising a body extending longitudinally between a proximal end region and a distal end region, the support catheter being able to insert, position and deploy the endoprosthesis in the blood flow conduit, the support catheter comprising releasable means for retaining the endoprosthesis on the support catheter.

Such a device applies to the positioning, in a blood vessel, of a tubular endoprosthesis, commonly referred to as a “stent”, or of a tubular endovalve comprising a tubular endoprosthesis and a valve attached to the endoprosthesis.

A device of the aforementioned type is described in FR-A-2 863 160. This device comprises a tubular endoprosthesis and a hollow support catheter for supporting the endoprosthesis, comprising a body having a straight axis, the support catheter extending longitudinally between a proximal end region and a distal end region. The support catheter is able to insert, position and deploy the endoprosthesis in the blood vessel. The endoprosthesis is kept in its retracted state by means of filiform ties encircling it and by means of a rod for retaining these ties, provided inside the support catheter. Once the support catheter has been correctly positioned in the blood vessel, the ties are released and the endoprosthesis is gradually liberated until it reaches its deployed position, before withdrawing the support catheter from the blood vessel.

However, when the endoprosthesis has to be deployed in a curved blood vessel, for example at the aortic arch, the relative rigidity of the support catheter does not make it possible to keep the axis of the support catheter substantially parallel to the axis of the blood vessel close to the point of deployment of the endoprosthesis. In addition, the passage into the aortic arch may be difficult to achieve.

Since the deployment of the endoprosthesis is coaxial with respect to the axis of the support catheter, the positioning thereof in the blood vessel is difficult and laborious.

One object of the invention is therefore to provide a device for treating a blood vessel, which can be positioned more precisely in a curved blood vessel.

To this end, the invention relates to a treatment device of the aforementioned type, characterised in that at least the distal end region of the body of the support catheter is deformable between a substantially straight configuration and a curved configuration, and in that the device comprises control means for deforming said region.

In particular embodiments, the device according to the invention comprises one or more of the following features, taken individually or in all technically possible combinations:

the control means can be actuated from a proximal end of the support catheter;

the control means comprise a filiform element having a distal end attached to the distal end region of the body, the pulling and the displacement of the filiform element deforming the body between its straight configuration and its curved configuration;

the support catheter delimits, at least in a portion of the distal end region of the body, a conduit for guiding the filiform element;

the support catheter has, in its distal end region, a zone of differential elastic deformation that is able to undergo a differential longitudinal deformation along two opposite generatrices of the body between its straight configuration and its curved configuration, under the action of the control means;

the body of the support catheter comprises, in said zone, a plurality of external slots which extend circumferentially on the body, each slot being deformable between a rest configuration corresponding to the straight configuration of the body, in which each slot has a first width, and a tensioned configuration corresponding to the curved configuration of the body, in which each slot has a width smaller than the first width;

adjacent slots open into one and the same angular sector of the support catheter taken with respect to the axis of the support catheter;

adjacent slots are offset angularly from one another with respect to the axis of the support catheter;

the support catheter comprises, in said distal end region, a spring that is elastically deformable, under the action of said control means, between a rest configuration corresponding to the straight configuration of the body and a tensioned configuration corresponding to the curved configuration of the body, the turns of the spring being joined on a first angular sector of the spring taken with respect to its axis, whereas, on a second angular sector different from the first angular sector of the spring, the turns of the spring are free;

the spring comprises guide members for guiding the filiform element, said guide members being attached to the turns;

adjacent guide members are arranged along one and the same generatrix of the spring; and

adjacent guide members are offset angularly from one another with respect to the axis of the support catheter.

The invention will be better understood on reading the following description which is given solely by way of example and with reference to the appended drawings, in which:

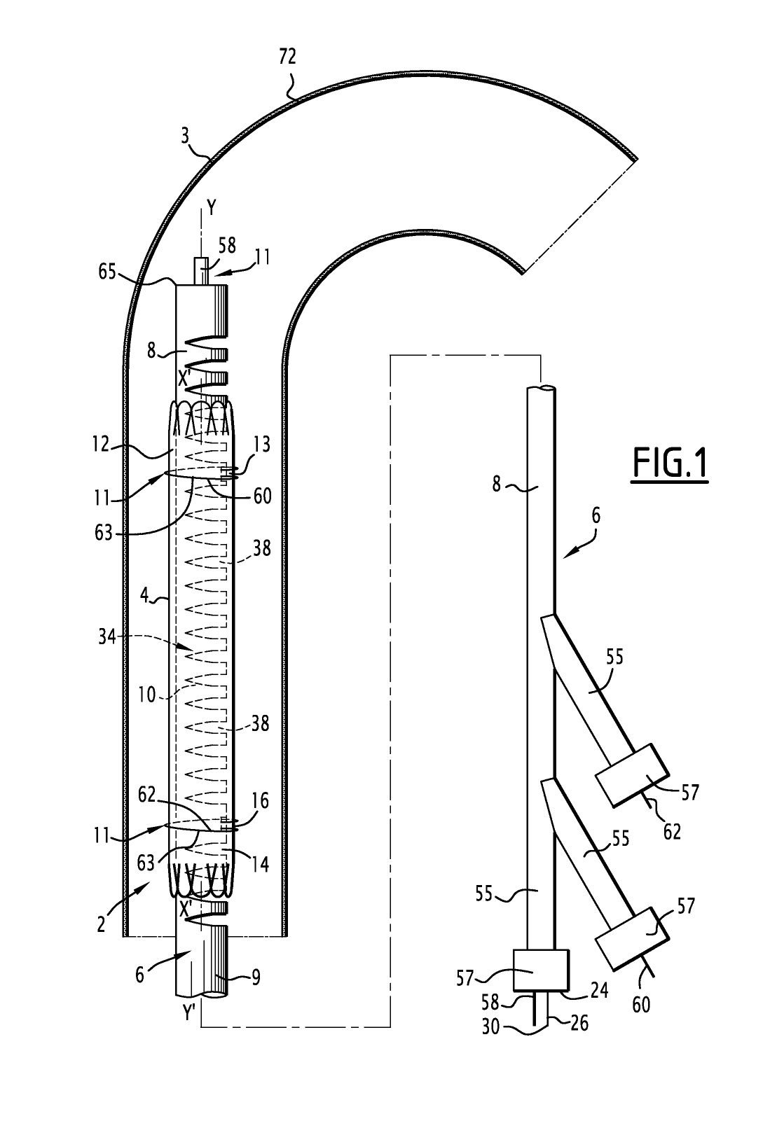

FIG. 1 is a schematic side view of a treatment device according to the invention as said device is being inserted into a blood vessel, the device comprising a support catheter in a straight configuration and an endoprosthesis kept in a retracted state;

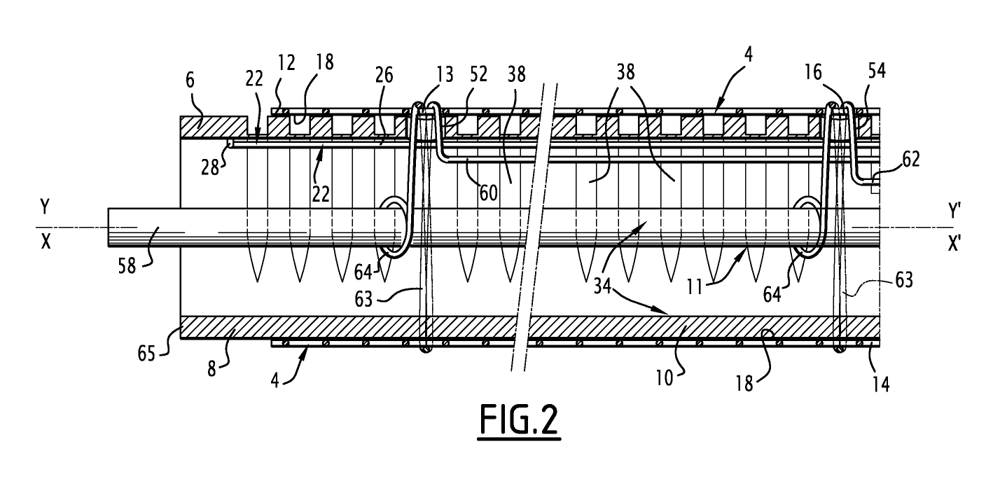

FIG. 2 is an enlarged view, taken in section along a median axial plane of the device of FIG. 1;

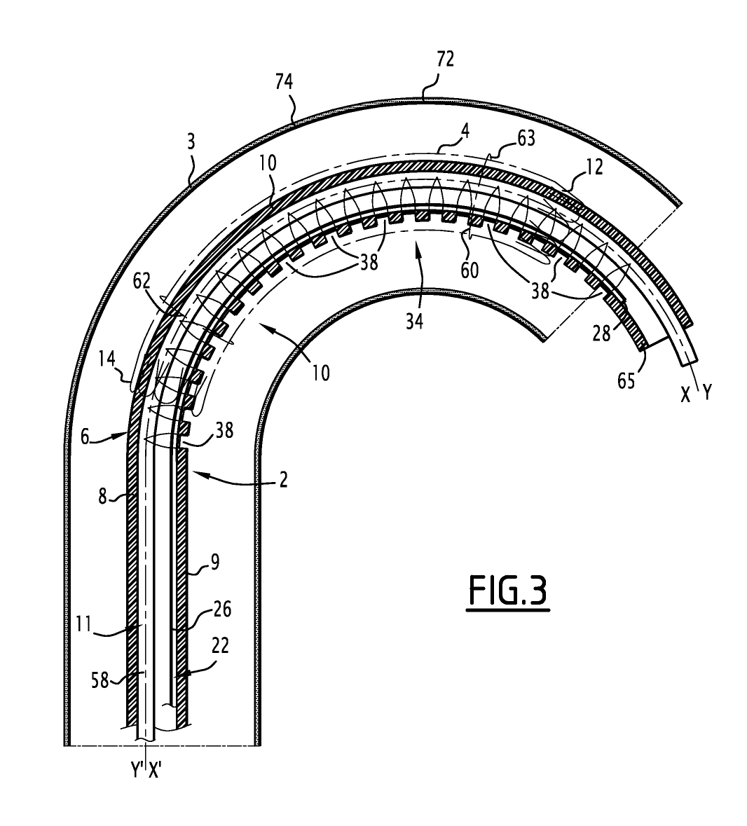

FIG. 3 is an enlarged view of part of the device of FIG. 1, the support catheter being in a curved configuration according to the invention;

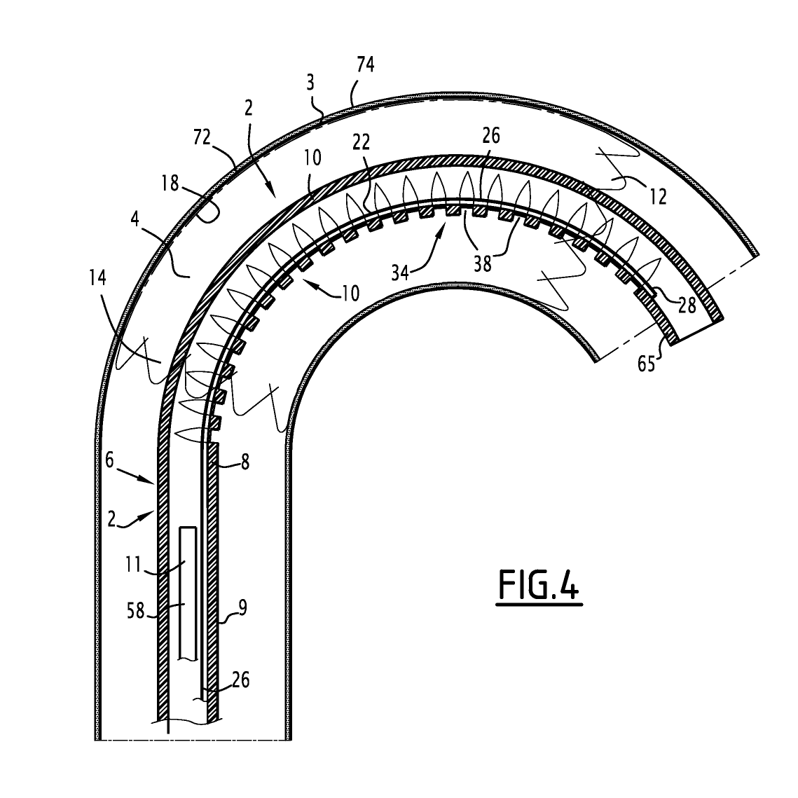

FIG. 4 is a view similar to FIG. 3, the endoprosthesis being in a dilated state;

FIG. 5 is an enlarged schematic side view of FIG. 4 illustrating a variant embodiment in which deformation slots are offset angularly; and

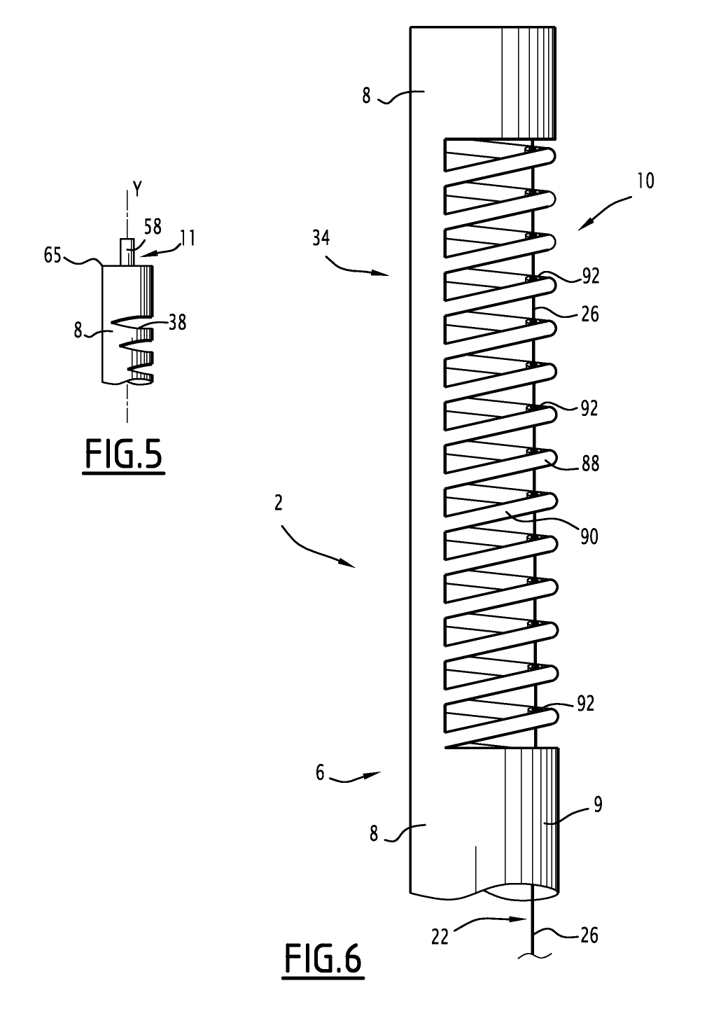

FIG. 6 is an enlarged schematic side view of a treatment device according to a second embodiment of the invention, in which the endoprosthesis is not shown.

FIGS. 1 to 4 show a device 2 for treating a blood vessel 3, comprising a tubular endoprosthesis 4 deployable between a retracted state and a dilated state, and a single support catheter 6 for supporting the endoprosthesis 4, said support catheter comprising a body 8 of axis Y-Y′ extending longitudinally between a proximal end region 9 and a distal end region 10.

The support catheter 6 is able to insert, position and deploy the endoprosthesis 4 in the blood vessel 3. To this end, the device 2 comprises releasable means 11 for retaining the endoprosthesis 4 on the body 8 of the support catheter 6.

The endoprosthesis 4 comprises a tubular trellis of axis X-X′ made from a material having spring properties. This endoprosthesis 4 is thus autoextensible.

As known per se, the endoprosthesis 4 is able to deform spontaneously from a compressed state, in which it has a small diameter (see FIG. 1), to a dilated state, in which it has a larger diameter (see FIG. 4), this dilated state constituting its rest state.

This endoprosthesis 4 is produced for example by braiding a single filament of a superelastic material, as described in European patent application EP-A-0 857 471.

The trellis of the endoprosthesis 4 defines, close to a distal end 12 of the endoprosthesis 4, a distal guide passage 13 for the retaining means 11 and, close to a proximal end 14 of the endoprosthesis 4, a proximal guide passage 16 for the retaining means 11.

Each guide passage 13, 16 is delimited by a trellis mesh of the endoprosthesis 4. The passages 13, 16 are located respectively on one and the same generatrix of the endoprosthesis 4 parallel to the axis X-X′.

The trellis has, at the ends 12, 14 of the endoprosthesis 4, folded filaments forming bends.

The endoprosthesis 4 delimits internally a blood flow conduit 18 of axis X-X′.

The proximal end region 9 of the body 8 is straight and rigid.

The distal end region 10 of the body 8 is deformable between a straight configuration, shown in FIG. 1, and a plurality of curved configurations, one of which is shown in FIGS. 3 and 4. In this curved configuration, the support catheter 6 is able to insert, position and deploy the endoprosthesis 4 in the curved blood vessel 3, such as for example in the aortic arch.

The device 2 also comprises control means 22 which can be actuated from a proximal end 24 of the support catheter and which are able to deform said distal end region 10 between its straight configuration and its curved configuration.

These control means 22 comprise a control wire 26 extending inside the body 8 of the support catheter 6 between a distal end 28 of the wire 26 (FIG. 2), attached to the distal end region 10, and a proximal end 30 of the wire 26 protruding out of the tube 6. The body 8 thus delimits a guide conduit for the control wire 26. The proximal end 30 is suitable for the operator to pull on the control wire 26. The pulling and the displacement of the control wire 26 towards the proximal end 24 of the support catheter 6 deforms the body 8 of the support catheter 6 from its straight configuration to its curved configuration. In the curved configurations, the measured minimum distance between the distal end 28 of the wire 26 and the proximal end region 9 is smaller than that measured in the straight configuration of the support catheter 6. This is because, as the control wire 26 is pulled and displaced towards the proximal end 24 of the support catheter 6, this distance decreases. The decrease in this distance determines the deformation of the distal end region 10.

The support catheter 6 in fact has, in its distal end region 10, a zone of differential elastic deformation 34 that is able to undergo a predetermined differential longitudinal deformation along two opposite generatrices of the body 8 of the support catheter 6, from its straight configuration to its curved configuration, and vice versa, under the action of the control wire 26.

As shown in FIGS. 1 to 4, the body 8 of the support catheter 6 delimits, in the zone 34, a plurality of external transverse slots 38, each extending circumferentially around part of the outer circumference of the body 8, advantageously around at least 30% of the outer circumference, more advantageously around at least 50% of the outer circumference, preferably around at least 60% of the outer circumference.

The slots 38 are parallel and spaced apart longitudinally. Each slot 38 is deformable between a rest configuration of maximum width corresponding to the straight configuration of the body 8 and a tensioned and shrunk configuration of minimum width corresponding to the curved configuration of the body 8, as shown in particular in FIGS. 3 and 4.

The slots 38 open into one and the same angular sector of the support catheter 6, taken with respect to the axis Y-Y′ of the support catheter 6, that is to say over a longitudinal portion of the circumference of the body 8. The distal end 28 of the control wire 26 is itself attached to the body 8 in this same angular sector distally with respect to the slots 38. In this way, the pulling and the displacement of the control wire 26 towards the proximal end 24 of the support catheter 6 deforms the slots 38 from their rest configuration to their tensioned configuration so that the distal end region 10 curves in a predetermined direction with a predetermined amplitude from its straight configuration to its curved configuration. The deformation of the body 8 in a predetermined direction is the direct consequence of the deformation of the slots 38 in one and the same angular sector of the body 8.

In this assembly, the endoprosthesis 4 is mounted coaxially around the distal end region 10 of the support catheter 6. The support catheter 6 is inserted in the conduit 18.

The internal diameter of the body 8 of the support catheter 6 is suitable for slipping the latter onto a filiform surgical guide (not shown) installed on the patient, prior to the placement of the endoprosthesis 4 in the blood vessel 3 of this patient.

Distal 52 and proximal 54 retaining openings, offset longitudinally, are formed laterally in the body 8, in its distal end region 10.

The distance separating the distal retaining opening 52 from the proximal retaining opening 54 is substantially equal to the length of the endoprosthesis 4 in its retracted state, taken along the axis X-X′. The distal retaining opening 52 extends opposite the distal guide passage 13, and the proximal retaining opening 54 extends opposite the proximal guide passage 16.

As described in the application FR-A-2 863 160 by the applicant, the support catheter 6 moreover comprises hollow branches 55 close to its proximal end 24. These branches 55 communicate with the interior of the body 8 of the support catheter 6. A control passage 56 is formed at a free end 57 of each branch 55.

The releasable means 11 for retaining the endoprosthesis 4 comprise a retaining rod 58 and distal 60 and proximal 62 retaining wires.

The retaining rod 58 is arranged in the body 8. The length of the rod 58 is greater than or equal to the distance between the distal retaining opening 52 and the proximal end 24 of the support catheter 6.

The rod 58 is able to move in translation along the axis Y-Y′, between a retaining position in which the rod 58 is located opposite the two retaining openings 52 and 54, an intermediate position in which the rod 58 is located opposite the proximal retaining opening 54 and at a distance from the distal retaining opening 52, and a released position in which the rod 58 is located at a distance from the two retaining openings 52 and 54.

Each of the retaining wires 60, 62 forms, around the endoprosthesis 4, a retaining loop 63 and comprises an end hoop 64 in which the rod 58 is engaged when the rod 58 is in its retaining position. The retaining loop 63 fixes the endoprosthesis 4 to the body 8 of the support catheter 6. Furthermore, the active length of the retaining loop 63 is variable, so that it controls the deployment of the endoprosthesis 4 with respect to the support catheter 6.

As will be described below, the retaining loop 63 is extensible between a configuration of keeping the endoprosthesis 4 in its retracted state against the support catheter 6, in which it has a minimum diameter, and a released configuration of the endoprosthesis 4, in which it has a maximum diameter.

A control end of each of the retaining wires 60, 62 is engaged outside the support catheter 6, the length of this portion being variable and controlling the length of the retaining loop 63.

Thus, the displacement of the retaining wires 60, 62 with respect to the support catheter 6, towards the proximal end 24 of the support catheter 6, causes a corresponding reduction in the active length of the retaining loop 63 and consequently the clamping of the endoprosthesis 4 against the support catheter 6, at the retaining loop 63.

When the endoprosthesis 4 is in its retracted state against the support catheter 6, the wires 60, 62 are kept tensioned.

Conversely, the displacement of the proximal ends of the wires 60, 62 with respect to the support catheter 6 towards the distal end 65 of the body 8 of the support catheter 6 causes an increase in the active length of the retaining loop 63 radially away from the axis Y-Y′ of the body 8 and away from the distal guide passage 13. This extension of the loop 63 permits the deployment of the endoprosthesis 4 away from the body 8 of the support catheter 6, inside the retaining loop 63.

The mode of operation of the treatment device according to the invention will now be described by way of example.

In a first stage, the device 2 is kept in its packaging (not shown), with the endoprosthesis 4 in a deployed state similar to that shown in FIG. 4 and the support catheter 6 in its straight configuration.

In these configurations, the retaining rod 58 is in its retaining position. The distal and proximal retaining wires 60 and 62 are engaged in the rod 58 and in the trellis of the endoprosthesis 4.

This packaging preserves the mechanical properties of the endoprosthesis 4, particularly when the tubular trellis thereof is embedded in an extensible and sealed film, such as an elastomer.

In a second stage, the surgeon extracts the device from its packaging. He implants a surgical guide (not shown) which extends in the blood vessel 3 from the external point of introduction to the zone of the vessel 3 in which the tubular endoprosthesis 4 is to be implanted.

As shown in FIGS. 1, 3 and 4, this vessel 3 has a bend 72 close to the point of implantation 74 of the endoprosthesis 4.

In a third stage, with a view to implanting the endoprosthesis 4 in the blood vessel 3, the surgeon displaces the control portion of each retaining wire 60, 62 towards the proximal end 24 of the support catheter 6. The active length of the retaining loops 63 decreases so that the endoprosthesis 4 is retracted against the body 8 and securely fixed relative to the body 8, coaxially with respect to the body 8 of the support catheter 6.

In addition, the control wire 26 is still slack so that the distal end region 10 remains in its straight configuration, as illustrated in FIG. 1. The endoprosthesis 4 is thus introduced along a straight portion of the blood vessel 3.

In some cases, and in order to maintain a minimum radial size, a sleeve (not shown) is arranged around the endoprosthesis 4 prior to this introduction and removed once the introduction has been carried out.

In a fourth stage, when the distal end 65 of the body 8 arrives at the bend 72, the surgeon exerts a gradual pulling action on the control wire 26 and thus displaces the wire 26 towards the proximal end 24 of the support catheter 6, so as to deform the distal end region 10 of the body 8 into one of its curved configurations. During this displacement of the control wire 26, the distal end 28 of the wire 26 moves closer to the proximal end region 9 of the body 8.

FIGS. 3 and 4 show the distal end region 10 after deformation by the control wire 26. As shown in FIGS. 3 and 4, the axis Y-Y′ of the body 8 of the support catheter 6 and the axis X-X′ of the endoprosthesis 4 are, at least at the point of implantation 74, substantially coaxial with the curved axis of the bend 72 of the vessel 3.

Once the endoprosthesis 4 has been positioned at the point of implantation 74, the surgeon can choose to deploy first one or the other of the ends 12, 14 of the endoprosthesis 4. The deployment of the distal end 12 will be described by way of example.

First of all, the surgeon displaces the control portion of the wire 60 towards the distal end 65 of the body 8 of the support catheter 6. As a result, the active length of the retaining loop 63 increases.

The trellis of the endoprosthesis 4 then deforms spontaneously from the compressed state shown in FIG. 1 to the deployed state shown in FIG. 4.

If the surgeon is not satisfied with the positioning of the distal end 12 of the endoprosthesis 4 when the latter is deployed, he reduces the active length of the retaining loop 63 by pulling on the control portion of the wire so as to compress the endoprosthesis 4 against the support catheter 6. The endoprosthesis 4 is then displaced to a more satisfactory position.

In an analogous manner, the surgeon then deploys the proximal end 14 of the endoprosthesis 4, by means of the proximal retaining wire 62.

When the surgeon is satisfied with the positioning of the distal end 12 of the endoprosthesis 4, he displaces the retaining rod 58 from its retaining position to the intermediate position. During this displacement, the hoop 64 of the distal retaining wire 60 is freed from the rod 58.

The surgeon then pulls the control portion so as to cause the distal retaining wire 60 to exit through the control branch 55.

The distal end 12 of the endoprosthesis 4 is then irreversibly fixed in the blood vessel 3.

The surgeon carries out the same procedure for the proximal end 14 of the endoprosthesis 4.

The support catheter 6 according to the invention is thus suitable for a precise positioning of the endoprosthesis 4 in the bend 72 or beyond the bend 72, such as for example in or beyond the aortic arch.

As a variant, the endoprosthesis 4 is not mounted coaxially on the support catheter 6 but rather at the side of the support catheter 6, along a generatrix of the support catheter 6.

As a variant, the body 8 is attached to a separate tube secured to the support catheter 6. This is for example a tube adhesively bonded against the support catheter 6 or adhesively bonded inside the support catheter 6. The support catheter 6 then receives the retaining means 11 whereas the body 8 receives the control wire 26.

Also as a variant, the distal and proximal openings 52 and 54 are arranged on opposite generatrices, on each side of the axis Y-Y′ of the body 8.

As a further variant, the endoprosthesis 4 is not autoextensible. The support catheter 6 is then equipped with an inflatable balloon arranged around the support catheter 6, between the support catheter 6 and the endoprosthesis 4, and able to deform the endoprosthesis 4 plastically between an insertion state of small diameter and a deployed state of large diameter.

In another variant, the distal end region 10 comprises a rigid and straight distal portion and a proximal portion that is deformable between the straight configuration and the curved configuration.

As a further variant, the support catheter 6 comprises two zones of differential elastic deformation 34 that are spaced apart axially. The body 8 of the support catheter 6 thus forms for example a U-shape or even a spiral loop in its curved configuration.

In a variant shown in FIG. 5, the deformation zone 34 is also able to deform by rotation about the axis Y-Y′. To this end, the adjacent slots 38 are offset angularly from one another about the axis Y-Y′. Each slot 38 is for example located in an angular sector that is offset angularly about the axis Y-Y′ with respect to the angular sector of an adjacent slot 38, each slot 38 also being offset axially along the axis Y-Y′ with respect to the adjacent slots 38. The angular offset is for example constant. The body 8 of the support catheter 6 is then deformed into part of a spiral when it is in its curved configuration.

FIG. 6 shows a second embodiment of the invention. Only the differences compared to the first embodiment will be described below.

The body 8 of the support catheter 6 comprises, in the distal end region 10, an elastically deformable portion formed by a spring 88. The spring 88 is deformable, under the action of the control wire 26, between a rest configuration corresponding to the straight configuration of the body 8 and a tensioned configuration corresponding to the curved configuration of the body 8.

The spring 88 is of axis Y-Y′. The turns 90 of the spring 88 are joined in a first angular sector of the spring 88 taken with respect to the longitudinal axis of the spring 88, whereas the turns 90 are free along a second angular sector different from the first angular sector. The distal end 28 of the control wire 26 is attached to the distal end of the spring 88, in the second angular sector of the spring 88.

The spring 88 thus has a zone 34 of differential elastic deformation that is able to undergo a differential elastic longitudinal deformation along two opposite generatrices of the spring 88, between its rest configuration and its tensioned configuration, under the action of the control wire 26.

The displacement of the control wire 26 compresses the spring 88 to its tensioned configuration in which the turns have, in the second angular sector, a smaller spacing than in the rest configuration, whereas in the first angular sector the spacing between the turns 90 remains substantially constant with respect to the rest configuration.

Furthermore, the turns 90 of the spring 88 comprise, in the second angular sector, loops 92 delimiting a guide passage in which the control wire 26 is received.

During the pulling and the displacement of the control wire 26 by the surgeon towards the proximal end 24 of the support catheter 6, the spring 88 is deformed by a predetermined amplitude and in a predetermined direction, so that the distal end region 10 is moved from its straight configuration to its curved configuration.

As a variant, the loops 92 are rings welded onto the spring 88.

As a variant, the first angular sectors of the spring 88 are offset angularly with respect to the axis Y-Y′, in the same way as the slots 38 are offset in the embodiment of FIG. 5. Similarly, the guide members 92 are offset angularly from one another. The body 8 of the support catheter 6 is thus deformed into part of a spiral when it is in its curved configuration.

1. Device for treating a blood flow conduit, of the type comprising:
at least one endoprosthesis deployable between a retracted state and a dilated state;
a support catheter for supporting the endoprosthesis, the support catheter comprising a body extending longitudinally between a proximal end region and a distal end region, the support catheter being able to insert, position and deploy the endoprosthesis in the blood flow conduit, the support catheter comprising releasable means for retaining the endoprosthesis on the support catheter, characterised in that at least the distal end region of the body of the support catheter is deformable between a substantially straight configuration and a curved configuration, and in that the device comprises control means for deforming said distal end region between its straight configuration and its curved configuration.

2. Device according to claim 1, in which the control means can be actuated from a proximal end of the support catheter.

3. Device according to claim 1, in which the control means comprise a filiform element having a distal end attached to the distal end region of the body, the pulling and the displacement of the filiform element deforming the body between its straight configuration and its curved configuration.

4. Device according to claim 3, in which the support catheter delimits, at least in a portion of the distal end region of the body, a conduit for guiding the filiform element.

5. Device according to claim 1, in which the support catheter has, in its distal end region, a zone of differential elastic deformation that is able to undergo a differential longitudinal deformation along two opposite generatrices of the body between its straight configuration and its curved configuration, under the action of the control means.

6. Device according to claim 5, in which the body of the support catheter comprises, in said zone, a plurality of external slots which extend circumferentially on the body, each slot being deformable between a rest configuration corresponding to the straight configuration of the body, in which each slot has a first width, and a tensioned configuration corresponding to the curved configuration of the body, in which each slot has a width smaller than the first width.

7. Device according to claim 6, in which adjacent slots open into one and the same angular sector of the support catheter taken with respect to the axis (Y-Y′) of the support catheter.

8. Device according to claim 6, characterised in that adjacent slots are offset angularly from one another with respect to the axis (Y-Y′) of the support catheter (6).

9. Device according to claim 5, in which the support catheter comprises, in said distal end region, a spring that is elastically deformable, under the action of said control means, between a rest configuration corresponding to the straight configuration of the body and a tensioned configuration corresponding to the curved configuration of the body, the turns of the spring being joined on a first angular sector of the spring taken with respect to its axis, whereas, on a second angular sector different from the first angular sector of the spring, the turns of the spring are free.

10. Device according to claim 9, in which the control means comprise a filiform element having a distal end attached to the distal end region of the body, the pulling and the displacement of the filiform element deforming the body between its straight configuration and its curved configuration, in which the spring comprises guide members for guiding the filiform element, said guide members being attached to the turns.

11. Device according to claim 10, characterised in that adjacent guide members are arranged along one and the same generatrix of the spring.

12. Device according to claim 11, characterised in that adjacent guide members are offset angularly from one another with respect to the axis (Y-Y′) of the support catheter.
